(19) United States
(12) Patent Application Publication
(10) Pub. No.: US 2007/0149457 A1
(43) Pub. Date: Jun. 28, 2007

(54) Stable solid forms of enterostatin

(76) Inventors: Byron Rubin, Honeoye Falls, NY (US); David T. Jonaitis, Lafayette, IN (US); Stephan D. Parent, West Lafayette, IN (US)

Correspondence Address: JONES DAY, 222 EAST 41ST ST, NEW YORK, NY, 10017, US

(21) Appl. No.: 11/638,047

(22) Filed: Dec. 12, 2006

Related U.S. Application Data
(60) Provisional application No. 60/750,207, filed on Dec. 13, 2005.

(51) Int. Cl. A61K 38/31

(52) U.S. Cl. 514/12; 530/311

(57) ABSTRACT

The present invention provides co-complexes of enterostatin that can display advantageous hygroscopicity, advantageous stability, or both. The co-complexes of enterostatin can be useful for the manufacture of an pharmaceutical product comprising enterostatin.

This application claims the benefit of priority of U.S. provisional application No. 60/750,207, filed Dec. 13, 2005, the contents of which are hereby incorporated by reference in their entireties.

### 1. FIELD OF THE INVENTION

The present invention provides novel solid forms of peptides that modulate F₁-ATPase activity, including novel solid forms of enterostatin. In certain embodiments, the invention provides solid forms that have reduced hygroscopicity compared to the conventional forms of the peptides. Thus, the invention also provides the use of the solid forms of the invention, for example, in and for the manufacture of a pharmaceutical formulations. The solid forms and formulations can be used for the treatment or prevention of certain metabolic conditions including those related to enterostatin activity or F₁-ATPase activity, such as obesity and diabetes.

### 2. BACKGROUND OF THE INVENTION

Obesity is a complex condition that is increasingly affecting the population worldwide. According to the World Health Organization, in 1995 there were an estimated 200 million obese adults worldwide and another 18 million under-five children classified as overweight. As of 2000, the number of obese adults had increased to over 300 million. See Formiguera et al., 2004, Best Practice & Research Clinical Gastroenterology, 18:6, 1125-1146.

Overweight or obesity has been shown to increase risk for several diseases and health conditions, including hypertension, dyslipidemia (high total cholesterol or high levels of triglycerides), type II diabetes, coronary heart disease, stroke, gallbladder disease, osteoarthritis, sleep apnea and respiratory problems and some cancers (for example, endometrial, breast, and colon). See, e.g., U.S. National Center for Chronic Disease Prevention and Health Promotion. Its health consequences range from increased risk of premature death to serious chronic conditions that reduce the overall quality of life.

Various therapies have been proposed or tested for the modulation of physiological processes that might lead to conditions such as overweight or obesity. See Orzano et al., 2004, J. Am. Board Fam. Pract. 17(5):359-69. One of these is enterostatin.

Enterostatin is a peptide that has shown promise in modulating dietary fat preference in rodents. See, e.g., Erlanson-Albertsson et al., 1991, Physiol. Behav. 49:1191-1194; Okada et al., 1991, Physiol. Behav. 49:1185-1189; Shargill et al., 1991, Brain Res. 544:137-140. Enterostatin is generated by tryptic activation of procolipase in the intestine or stomach to generate colipase. Colipase binds and activates the enzyme lipase to metabolize fats in the intestine. The propeptide enterostatin is believed to reduce dietary fat preference in mammals as demonstrated in rodent studies. See, Erlanson-Albertsson et al., 1991, Okada et al., 1991, Physiol. Behav. 49:1185-1189, Shargill et al., 1991. Accordingly, studies of decreasing appetite in mammals by administering an effective amount of an enterostatin peptide have been reported. See, Erlanson-Albertsson, 1996, U.S. Pat. No. 5,494,894. Human studies concerning endogenous enterostatin have been reported. See e.g., Prasad et al., 1999, J. Clin. Endocrinol. Metab. 84:937-941; Kovacs et al., 2003, British J. Nutrition 90:207-214.

In developing novel methods of administering enterostatin, it was discovered that conventional forms of enterostatin can take on too much water in ambient or storage conditions for the efficient manufacture of an enterostatin pharmaceutical product. Conventional forms of enterostatin that absorb too much water can degrade over time and can be difficult to measure and administer reproducibly. Those of skill in the art will recognize that the hygroscopicity of conventional forms of enterostatin can be too great for efficient manufacture, storage and use in conventional pharmaceutical tablets or capsules.

Stable forms of enterostatin are needed for the manufacture of pharmaceuticals comprising enterostatin useful for the modulation of food intake and the treatment or prevention of metabolic conditions including those associated with enterostatin or F₁-ATPase activity, such as obesity or diabetes.

### 3. SUMMARY OF THE INVENTION

The present invention provides novel, stable forms of peptides such as enterostatin. The stable forms of the invention can display reduced hygroscopicity and increased stability in ambient conditions or in storage conditions. Accordingly, the novel, stable forms of the invention are useful in and for the manufacture of pharmaceutical products. The novel, stable forms can be used for the treatment or prevention of any condition or disorder for which the peptide itself is useful. For instance, the novel, stable forms of the invention can be used for the treatment or prevention of conditions related to enterostatin or F₁-ATPase activity, such as described in U.S. provisional application No. 60/750,208, filed Dec. 13, 2005, entitled “Methods of Treating Obesity Using Enterostatin,” the contents of which are hereby incorporated by reference in its entirety. Exemplary disorders or conditions related to enterostatin or F₁-ATPase activity include, but are not limited to, overweight, obesity, metabolic disorders, hypertension, lipid related disorders, and type II diabetes.

The present invention is based, in part, on the discovery of stable forms of peptides with enterostatin or F₁-ATPase activity that can display reduced hygroscopicity. In particular, the present invention is based, in part, on the discovery of forms peptides such as enterostatin that can have reduced hygroscopicity and increased stability.

In aspects of the invention, the present invention provides stable forms that are in the form of a co-complex. For the present invention, unless stated otherwise, a co-complex comprises a host molecule and a guest molecule. The terms “host” and “guest” are used herein conveniently to refer to components of a co-complex. Unless specified otherwise, they can be used interchangeably and are not intended to describe any particular aspect of the components such as the size of the components, how they interact, etc. In particular, although a peptide component of a co-complex of the invention might be referred to as a “host” herein, it could just as easily be described as a “guest.”

The co-complex can be in any form for a co-complex known to those of skill in the art. For instance, the co-complex can be in liquid, solid or semi-solid form. In preferred embodiments, the co-complex is in a solid form as such solid forms can be useful for the manufacture of a pharmaceutical product. In certain embodiments, the solid form has crystalline properties. In particular embodiments, the solid form is crystalline.

In aspects of the invention, the host can be any peptide with enterostatin or F₁-ATPase activity. In certain embodiments, the host is an enterostatin peptide. In particular embodiments, the host is an enterostatin peptide having a sequence selected from the group consisting of consisting of APGPR (SEQ ID NO:1), VPDPR (SEQ ID NO:2) and VPGPR (SEQ ID NO:3).

The guest can be any guest molecule capable of forming a co-complex with the host. In preferred embodiments, the guest is a molecule capable of reducing the hygroscopicity of the host, increasing the stability of the host or both. In certain embodiments, the guest is an organic acid or an organic base. In certain embodiments, the guest is selected from the group consisting of adipic acid, piperazine, hippuric acid, piperazine, camphoric acid, gentisic acid and naphthoic acid.

In one aspect, the present invention provides novel, stable co-complexes that comprise a host of the invention and a guest of the invention. In certain embodiments, the host can be in a neutral or a salt form. In certain embodiments, the guest can be in a neutral or salt form. In certain embodiments, the host and the guest can be in a neutral or salt form. Preferred salts of the host include chlorides, acetates, sulfates and phosphates. Preferred salts of the guest include sodium, potassium, calcium, magnesium and ammonium salts.

In another aspect, the present invention provides novel, stable salts of a peptide with enterostatin or F₁-ATPase activity. In certain embodiments, the peptide is an enterostatin peptide. In particular embodiments, the peptide is an enterostatin peptide having a sequence selected from the group consisting of consisting of APGPR (SEQ ID NO:1), VPDPR (SEQ ID NO:2) and VPGPR (SEQ ID NO:3). Preferred salts include chlorides, acetates, sulfates and phosphates. In certain embodiments, the novel, stable salt of the peptide can be co-complexed with a guest molecule, as described above. A preferred guest molecule can be selected from the group consisting of acid, piperazine, hippuric acid, piperazine, camphoric acid, gentisic acid and naphthoic acid.

In another aspect, the present invention provides a crystalline, non-hygroscopic form of a peptide with enterostatin or F₁-ATPase activity. In certain embodiments, the peptide is an enterostatin peptide. In particular embodiments, the peptide is an enterostatin peptide having a sequence selected from the group consisting of consisting of APGPR (SEQ ID NO:1), VPDPR (SEQ ID NO:2) and VPGPR (SEQ ID NO:3). The crystalline form of the peptide can be any form of the peptide having crystalline properties. The crystalline properties can be any crystalline properties known to those of skill in the art including, for example, X-ray diffraction, X-ray powder diffraction, birefringence, Raman scattering and the like. In certain embodiments, the present invention provides an enterostatin crystal. In certain embodiments, the crystalline, non-hygroscopic form comprises a guest molecule as described above. In particular embodiments, the guest molecule is selected from the group consisting of adipic acid, piperazine, hippuric acid, piperazine, camphoric acid, gentisic acid and naphthoic acid.

In a particular aspect, the present invention provides an enterostatin co-complex comprising a peptide with enterostatin or F₁-ATPase activity and a naphthoic acid. The naphthoic acid can be any naphthoic acid known to those of skill in the art that is capable of forming a co-complex with the enterostatin. In preferred embodiments, the naphthoic acid is according to formula (I): 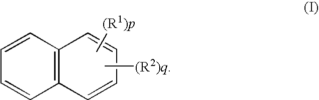
In formula (I), each p and q is independently an integer from 1 to 3; each R¹ is independently hydroxy or hydrogen; and each R² is independently carboxyl. Advantageously, in formula (I), each R¹ or R² independently can be on either ring of formula (I). Preferred naphthoic acids include 1-hydrox-2-napthoic acid.

In another aspect, the present invention provides solvates of any of the above stable forms of enterostatin. The solvent of the solvate can be any solvent known to those of skill in the art. Preferred solvents are physiologically acceptable. In particular embodiments, the solvate is a hydrate. Thus, the invention further provides crystalline solvates, e.g. crystalline hydrates, as well as crystalline salt solvates, e.g. crystalline salt hydrates.

In yet another aspect, the present invention provides anhydrous or low water-containing forms of enterostatin. In certain embodiments, the enterostatin is in the form of an enterostatin peptide. In certain embodiments, the enterostatin is in the form of a co-complex of an enterostatin. Exemplary anhydrous or low water-containing forms of enterostatin are described herein.

In a further aspect, the present invention provides a pharmaceutical composition comprising co-complex or solid form of the invention and one or more pharmaceutically acceptable carriers, excipients or diluents. In certain embodiments, the pharmaceutical compositions are packaged in unit dosage forms.

In another aspect, the present invention provides methods of treating or preventing a metabolic condition or disorder. In certain embodiments, the condition or disorder is associated with F₁-ATPase activity or enterostatin activity. In particular embodiments, the condition is associated with enterostatin deficiency. The methods comprise the step of administering an effective amount of a co-complex of the invention, a solid form of the invention, or a pharmaceutical composition of the invention, to a subject in need thereof. The methods are useful for the treatment or prevention of any condition associated with enterostatin including, but not limited to, overweight or obesity.

### 4. BRIEF DESCRIPTION OF THE DRAWINGS

FIG. 1 provides differential scanning calorimetry (DSC) measurements of an enterostatin acetate, 1-hydroxy-2-naphthoic acid co-complex of the invention;

FIG. 2 provides a Raman spectrum of an enterostatin acetate, 1-hydroxy-2-naphthoic acid co-complex of the invention;

FIG. 3 provides moisture balance of an enterostatin acetate, 1-hydroxy-2-naphthoic acid co-complex of the invention; and

FIG. 4 provides an X-ray powder diffraction (XRPD) analysis of an enterostatin acetate, gentisic acid co-complex of the invention.

### 5. DETAILED DESCRIPTION OF THE INVENTION

### 5.1 Definitions

When referring to the compounds and complexes of the invention, the following terms have the following meanings unless indicated otherwise.

The term “enterostatin” encompasses the propeptide of procolipase, as is known to those of skill in the art. Exemplary enterostatins have an amino acid sequence selected from the group consisting of APGPR (SEQ ID NO:1), VPDPR (SEQ ID NO:2) and VPGPR (SEQ ID NO:3). In a preferred embodiment, the enterostatin has an amino acid sequence of APGPR (SEQ ID NO:1).

“Pharmaceutically acceptable salt” refers to any salt of a compound of this invention which retains its biological properties and which is not toxic or otherwise undesirable for pharmaceutical use. Such salts may be derived from a variety of organic and inorganic counter-ions well known in the art and include. Such salts include: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) salts formed when an acidic proton present in the parent compound either (a) is replaced by a metal ion, e.g., an alkali metal ion, an alkaline earth ion or an aluminum ion, or alkali metal or alkaline earth metal hydroxides, such as sodium, potassium, calcium, magnesium, aluminum, lithium, zinc, and barium hydroxide, ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Salts further include, by way of example only, sodium, potassium, calcium, magnesium, ammonium, tetraalkylammonium and the like, and when the compound contains a basic functionality, salts of non-toxic organic or inorganic acids, such as hydrochloride, hydrobromide, tartrate, mesylate, besylate, acetate, maleate, oxalate and the like. The term “physiologically acceptable cation” refers to a non-toxic, physiologically acceptable cationic counterion of an acidic functional group. Such cations are exemplified by sodium, potassium, calcium, magnesium, ammonium and tetraalkylammonium cations and the like.

“Hygroscopicity” refers to sorption, implying an acquired amount or state of water sufficient to affect the physical or chemical properties of the substance (Eds. J. Swarbrick and J. C. Boylan, Encyclopedia of Pharmaceutical Technology, Vol. 10, p. 33).

“Solvate” refers to a compound of the present invention or a salt thereof, that further includes a stoichiometric or non-stoichiometric amount of solvent bound by non-covalent intermolecular forces. Where the solvent is water, the solvate is a hydrate.

It is to be understood that compounds having the same molecular formula but differing in the nature or sequence of bonding of their atoms or in the arrangement of their atoms in space are termed “isomers”. Isomers that differ in the arrangement of their atoms in space are termed “stereoisomers”.

The term “subject” refers to an animal such as a mammal, including, but not limited to, primate (e.g., human), cow, sheep, goat, horse, dog, cat, rabbit, rat, mouse and the like. In preferred embodiments, the subject is a human.

“Therapeutically effective amount” means an amount of a compound or complex or composition that, when administered to a subject for treating a disease, is sufficient to effect such treatment for the disease. A “therapeutically effective amount” can vary depending on, inter alia, the compound, the disease and its severity, and the age, weight, etc., of the subject to be treated.

“Treating” or “treatment” of any disease or disorder refers, in one embodiment, to ameliorating the disease or disorder (i.e., arresting or reducing the development of the disease or at least one of the clinical symptoms thereof) that exists in a subject. In another embodiment, “treating” or “treatment” refers to ameliorating at least one physical parameter, which may be indiscernible by the subject. In yet another embodiment, “treating” or “treatment” refers to modulating the disease or disorder, either physically (e.g., stabilization of a discernible symptom) or physiologically (e.g., stabilization of a physical parameter) or both. In yet another embodiment, “treating” or “treatment” refers to delaying the onset of the disease or disorder.

It is to be understood that compounds having the same molecular formula but differing in the nature or sequence of bonding of their atoms or in the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, when it is bonded to four different groups, a pair of enantiomers is possible. An enantiomer can be characterized by the absolute configuration of its asymmetric center and is designated (R) or (S) according to the rules of Cahn and Prelog (Cahn et al., 1966, Angew. Chem. 78:413-447, Angew. Chem., Int. Ed. Engl. 5:385-414 (errata: Angew. Chem., Int. Ed. Engl. 5:511); Prelog and Helmchen, 1982, Angew. Chem. 94:614-631, Angew. Chem. Internat. Ed. Eng. 21:567-583; Mata and Lobo, 1993, Tetrahedron: Asymmetry 4:657-668) or can be characterized by the manner in which the molecule rotates the plane of polarized light and is designated dextrorotatory or levorotatory (i.e., as (+)- or (−)-isomers, respectively). A chiral compound can exist as either individual enantiomer or as a mixture thereof. A mixture containing equal proportions of enantiomers is called a “racemic mixture”.

In certain embodiments, the compounds of this invention may possess one or more asymmetric centers; such compounds can therefore be produced as the individual (R)-or (S)-enantiomer or as a mixture thereof. Unless indicated otherwise, for example by designation of stereochemistry at any position of a formula, the description or naming of a particular compound in the specification and claims is intended to include both individual enantiomers and mixtures, racemic or otherwise, thereof. Methods for determination of stereochemistry and separation of stereoisomers are well-known in the art. In particular embodiments, the present invention provides the stereoisomers of the compounds depicted herein upon treatment with base.

In certain embodiments, the compounds or co-complexes of the invention are “stereochemically pure.” A stereochemically pure compound or co-complex has a level of stereochemical purity that would be recognized as “pure” by those of skill in the art. Of course, this level of purity will be less than 100%. In certain embodiments, “stereochemically pure” designates a compound or co-complex that is substantially free of alternate isomers. In particular embodiments, the compound or co-complex is 85%, 90%, 91%, 92%, 93%, 94%, 95%, 96%, 97%, 98%, 99%, 99.5% or 99.9% free of other isomers.

The amino acid notations used herein for the twenty genetically encoded L-amino acids are conventional and are as follows:

| | One-Letter | Three Letter |
|---|---|---|
| Amino Acid | Abbreviation | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

As used herein, unless specifically delineated otherwise, the three-letter amino acid abbreviations designate amino acids in the L-configuration. Amino acids in the D-configuration are preceded with a “D-.” For example, Arg designates L-arginine and D-Arg designates D-arginine. Likewise, the capital one-letter abbreviations refer to amino acids in the L-configuration. Lower-case one-letter abbreviations designate amino acids in the D-configuration. For example, “R” designates L-arginine and “r” designates D-arginine.

Unless noted otherwise, when peptide or polypeptide sequences are presented as a series of one-letter and/or three-letter abbreviations, the sequences are presented in the N-terminal to C-terminal direction, in accordance with common practice.

In preferred embodiments, any peptide or amino acid of the invention is in the L form, unless otherwise indicated.

### 5.2 Embodiments of the Invention

The present invention provides novel, stable forms of peptides having F₁-ATPase activity or enterostatin activity. The stable forms can display advantageous hygroscopicity and/or advantageous stability. The stable forms are useful, for example, as pharmaceutical products, for the manufacture of pharmaceutical products and for long term storage of the peptides. In particular embodiments, the stable forms are useful as active ingredients for use in oral dosage forms including, but not limited to, single unit dosage forms such as tablets, capsules, cachets, dragees. The dosage forms do not necessarily use or are not necessarily made under anhydrous conditions, and the invention provides dosage forms that are made under conditions with some moisture.

In certain embodiments, the stable forms are co-complexes. According to the present invention, the co-complexes comprise a host molecule and a guest molecule. Host molecules and guest molecules are described in detail below.

In the stable forms, the components can be in neutral forms, one component can be in a salt form, or both components can be in a salt form. Exemplary salt forms are described in detail in the sections below.

Accordingly, in certain embodiments, the present invention provides stable, non-hygroscopic forms of enterostatin. In further embodiments, the present invention provides stable, non-hygroscopic forms of neutral enterostatin. In further embodiments, the present invention provides stable, non-hygroscopic forms of a salt of enterostatin.

In certain embodiments, the solid forms of the invention are non-hygroscopic. The term “non-hygroscopic” refers to a level of hygroscopicity considered low by those of skill in the art. For instance, the solid form can have a hygroscopicity considered acceptable to those of skill in the art for the manufacture or storage of a pharmaceutical. In certain embodiments, a non-hygroscopic solid form of the invention will absorb less than 10%, 9%, 8%, 7%, 6%, 5%, 4%, 3%, 2% or 1% water by weight in an atmosphere of normal humidity. In certain embodiments, a non-hygroscopic solid form of the invention will remain solid for at least 1, 2, 3, 4, 5, 10, 15 or 20 days at at least 25%, 50% or 75% humidity. In preferred embodiments, a solid form of the invention will remain solid for at least 4 or 10 days at at least 58% humidity. In certain embodiments, the solid forms will gain less than 35%, 30%, 25% or 20% water, by weight, when moved from 5% to 95% relative humidity under techniques known to those of skill in the art. In certain embodiments, the solid forms will lose less than 35%, 30%, 25% or 20% water, by weight, when moved from 95% to 5% relative humidity under techniques known to those of skill in the art. In certain embodiments, the solid forms will gain less than 35%, 30%, 25% or 20% water, by weight, when moved from 5% to 95% relative humidity, and they will lose less than 35%, 30%, 25% or 20% water, by weight, when moved from 95% to 5% relative humidity. In certain embodiments, the solid forms of the invention have a water content of from 0 to 20%, 0 to 15%, 0 to 10%, 0.5 to 6% or 0.5 to 5% on a weight basis.

The term “stable” refers to a form of enterostatin that is stable under routine manufacturing, storage or usage conditions known to those of skill in the art. In certain embodiments, a stable form will maintain at least 50%, 60%, 67%, 70%, 75%, 80%, 85% or 90% activity for at least 1, 2, 3, 4, 5, 10, 15 or 20 days, or for at least one, two, three of four months under routine storage conditions. In certain embodiments, a stable form will maintain at least 50%, 60%, 67%, 70%, 75%, 80%, 85% or 90% activity for one, two, three, four or six months, or one, two or three years at about 4° C. In certain embodiments, a stable form will maintain at least 50%, 60%, 67%, 70%, 75%, 80%, 85% or 90% activity for one, two, three, four or six months, or one, two or three years at ambient temperature.

In certain embodiments, the solid forms of the invention are crystalline. Crystalline solid forms of the invention have one or more crystalline property that would be recognized by those of skill in the art. For instance, crystalline solid forms of the invention can have on or more properties selected from the group consisting of birefringence, defined X-ray powder diffraction peaks, defined X-ray diffraction peaks or spots, Raman scattering, defined melting temperature, defined shape, or any other crystalline property known to those of skill in the art. In certain embodiments, the present invention provides crystalline forms of enterostatin peptides.

As the stable forms of the invention find use, for example, in and for the manufacture of pharmaceutical products, the present invention also encompasses solvates of the solid forms of the invention. As will be recognized by those of skill in the art, a solvate of a solid form of the invention comprises the solid form coordinated with one or more solvent molecules. In preferred embodiments, the solvent is pharmaceutically acceptable. In particularly preferred embodiments, the solvent is water, i.e. the solvate is a hydrate.

### 5.3 Enterostatin Co-complexes of the Invention

The enterostatin co-complexes of the invention comprise an enterostatin host molecule and an co-complex guest. The enterostatin and the guest can be in their neutral forms, in salt forms or in combinations thereof.

5.3.1.1 Host Molecule

The enterostatin can be any enterostatin known to those of skill in the art. The enterostatin can be from the same species as a subject to be treated, or the enterostatin can be from a different species. In preferred embodiments, the enterostatin is from the same species as the subject. Exemplary enterostatins include human, rat, mouse, porcine, canine and equine enterostatin.

In certain embodiments, the enterostatin is a full-length enterostatin. Exemplary enterostatins have an amino acid sequence selected from the group consisting of APGPR (SEQ ID NO:1), VPDPR (SEQ ID NO:2) and VPGPR (SEQ ID NO:3). The enterostatin co-complexes of the invention can comprise a single enterostatin, or they can comprise multiple enterostatins. Preferred is APGPR (SEQ ID NO:1). Methods of making the enterostatins are described in detail below.

In preferred embodiments, the enterostatin is substantially pure. In this context the term “substantially pure” indicates that the enterostatin is substantially free of contaminants not intended to be administered. Examples include peptide or amino acid contaminants and peptide synthesis reagents. In certain embodiments, the enterostatin is 85%, 90%, 91%, 92%, 93%, 94%, 95%, 96%, 97%, 98%, 99%, 99.5% or 99.9% pure. As discussed in detail the section below, enterostatin can be formulated for administration with one or more carriers, excipients or diluents.

The enterostatin can comprise free termini or blocked termini according to the judgment of those of skill in the art. Useful blocked termini include a C-terminal amide or an N-terminal acetyl, or both. In preferred embodiments, the enterostatin has free N- and C-termini.

The enterostatin can be in a neutral form, or in a salt form. The salt form can be any salt form known to those of skill in the art. Particularly useful salt forms are those that are coordinated with acetate, chloride, sulfate and phosphate. Acetate and chloride salts are preferred.

5.3.1.2 Guest Molecule

The guest can be any co-complex guest that can form a co-complex with enterostatin. Preferred guests are those that impart greater stability or reduced hygroscopicity, or both, to the co-complex. In certain embodiments, the guest is a neutral carboxylic acid or an organic base. In particular embodiments, the guest is selected from the group consisting of a naphthoic acid, a gentisic acid, a camphoric acid, a piperazine, a hippuric acid and adipic acid. In preferred embodiments the guest is naphthoic acid.

The guest can be in a neutral form, or in a salt form. The salt form can be any salt form known to those of skill in the art.

The enterostatin and guest can be in any ratio capable of forming a complex. In certain embodiments the ratio ofenterostatin to guest is 1:100, 1:10, 1:5, 1:4, 1:3, 1:2, 1:1.5, 1:1.25, 1:1.2, 1:1.1, 1.1:1, 1.2:1, 1.25:1, 1.5:1, 2:1, 3:1, 4:1, 5:1, 10:1 or 100:1. In particular embodiments, the ratio is about 1:1.

In certain embodiments, the guest is a naphthoic acid according to formula (I):

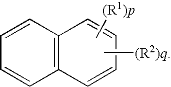

In formula (I), each R¹ or R² independently can be on either ring of formula (I). Each p and q is independently an integer from 1 to 3. In preferred embodiments, each p and q is 1. Each R¹ is independently hydroxy or hydrogen, and each R² is independently carboxyl. In a preferred embodiment, the naphthoic acid of formula (I) is 1-hydroxy-2-naphthoic acid.

In some embodiments, the co-complex comprises an acetate salt of an enterostatin having the sequence APGPR, and 1-hydroxy-2-naphthoic acid.

In some embodiments, the co-complex comprises a chloride salt of an enterostatin having the sequence APGPR, and 1-hydroxy-2-naphthoic acid.

5.3.1.3 Salts

Where a compound of the present invention, e.g. a host molecule or a guest molecule, is substituted with a basic moiety, an acid addition salt can be formed. The acid which can be used to prepare an acid addition salt includes preferably that which produces, when combined with the free base, a pharmaceutically acceptable salt, that is, a salt whose anion is non-toxic to a patient in the pharmaceutical doses of the salt. Pharmaceutically acceptable salts within the scope of the invention are those derived from the following acids: mineral acids such as hyrochloric acid, sulfuric acid, phosphoric acid and sulfamic acid; and organic acids such as acetic acid, citric acid, lactic acid, tartaric acid, malonic acid, methanesufonic acid, ethanesulfonic acid, benzenesulfonic acid, p-toluenesulfonic acid, cyclohexylsulfamic acid, quinic acid, and the like. The corresponding acid addition salts comprise the following: hydrohalides, e.g., hydrochloride and hydrobromide, sulfate, phosphate, nitrate, sulfamate, acetate, citrate, lactate, tartarate, malonate, oxalate, salicylate, propionate, succinate, fumarate, maleate, methylene-bis-.beta.-hydroxynaphthoates, gentisates, mesylates, isethionates and di-p-toluoyltartratesmethanesulfonate, ethanesulfonate, benzenesulfonate, p-toluenesulfonate, cyclohexylsulfamate and quinate, respectively.

According to a further feature of the invention, acid addition salts of the compounds of this invention can be prepared by reaction of the free base with the appropriate acid, by the application or adaptation of known methods. For example, the acid addition salts of the compounds of this invention can be prepared either by dissolving the free base in aqueous or aqueous-alcohol solution or other suitable solvents containing the appropriate acid and isolating the salt by evaporating the solution, or by reacting the free base and acid in an organic solvent, in which case the salt separates directly or can be obtained by concentration of the solution.

The acid addition salts of the compounds of this invention can be regenerated from the salts by the application or adaptation of known methods. For example, parent compounds of the invention can be regenerated from their acid addition salts by treatment with an alkali, e.g., aqueous sodium bicarbonate solution or aqueous ammonia solution.

Where a compound of the invention, e.g. a host molecule or a guest molecule, is substituted with an acid moiety, base addition salts can be formed. Pharmaceutically acceptable salts, including for example alkali and alkaline earth metal salts, within the scope of the invention are those derived from the following bases: sodium hydride, sodium hydroxide, potassium hydroxide, calcium hydroxide, aluminum hydroxide, lithium hydroxide, magnesium hydroxide, zinc hydroxide, ammonia, ethylenediamine, N-methyl-glucamine, lysine, arginine, omithine, choline, N,N′-dibenzylethylene-diamine, chloroprocaine, diethanolamine, procaine, N-benzylphenethylamine, diethylamine, piperazine, tris(hydroxymethyl)-aminomethane, tetramethylammonium hydroxide, and the like.

Metal salts of compounds of the present inventioncan be obtained by contacting a hydride, hydroxide, carbonate or similar reactive compound of the chosen metal in an aqueous or organic solvent with the free acid form of the compound. The aqueous solvent employed may be water or it may be a mixture of water with an organic solvent, preferably an alcohol such as methanol or ethanol, a ketone such as acetone, an aliphatic ether such as tetrahydrofuran, or an ester such as ethyl acetate. Such reactions are normally conducted at ambient temperature but they may, if desired, be conducted with heating.

Amine salts of compounds of the present invention can be obtained by contacting an amine in an aqueous or organic solvent with the free acid form of the compound. Suitable aqueous solvents include water and mixtures of water with alcohols such as methanol or ethanol, ethers such as tetrahydrofuran, nitrites, such as acetonitrile, or ketones such as acetone. Amino acid salts may be similarly prepared.

The base addition salts of the compounds of this invention can be regenerated from the salts by the application or adaptation of known methods. For example, parent compounds of the invention can be regenerated from their base addition salts by treatment with an acid, e.g., hydrochloric acid.

As well as being useful in themselves as active compounds, salts of compounds of the invention are useful for the purposes of purification of the compounds, for example by exploitation of the solubility differences between the salts and the parent compounds, side products and/or starting materials by techniques well known to those skilled in the art.

### 5.4 Preparation of Compounds and Co-Complexes of the Invention

The co-complexes can be prepared according to any method known to those of skill in the art, including those illustrated in the examples below.

Enterostatin can be prepared according to any technique apparent to those of skill. Exemplary techniques for the preparation of enterostatin are described in U.S. Pat. No. 5,494,894, the contents of which are hereby incorporated by reference in their entirety. In certain embodiments, enterostatin can be prepared synthetically, for example by solution phase or solid phase peptide synthesis. See Merrifield, 1963, J. Am. Chem. Soc. 85:2149; Fields et al., 1990, Int J Pept Protein Res. 35:161-214; Fields et al., 1991, Pept Res. 4:95-101; the contents of which are hereby incorporated by reference in their entireties. In further embodiments, enterostatin can be obtained from natural sources, recombinant sources or commercial sources.

The naphthoic acid of the complex can be prepared or obtained by any technique apparent to those of skill in the art. Useful synthetic techniques are described in Citterio et al., 1990, Synthesis 1990:142-144; Snider, 1996, Chem. Rev. 96:339-363; Dogan et al., 1998, Farmaco 53:462-7; Ji et al., 2004, Org Lett. 6:4551-3; Trost, 2002, Chem Pharm Bull (Tokyo) 50:1-14; Kim et al., 2002 Arch Pharm Res. 25:240-9; Boyfield et al., 1996, J Med Chem. 39:1946-8; the contents of which are incorporated by reference in their entireties. In certain embodiments, the naphthoic acid of the invention is obtained from commercial sources known to those of skill in the art.

The co-complex can be formed by any method of forming a co-complex known to those of skill in the art. Exemplary methods of forming co-complexes of the invention are described in the examples below. In certain embodiments, the enterostatin is contacted with the naphthoic acid in a suitable solvent under conditions in which a co-complex can form. The solution can then be evaporated by any evaporation technique to yield a co-complex of the invention.

The solvent can be any solvent in which the enterostatin and the naphthoic acid are soluble. In certain embodiments, the solvent is water, methanol, ethanol, isopropanol, hexafluoroisopropanol or a mixture thereof. Methanol and hexafluoroisopropanol are preferred.

The enterostatin and naphthoic acid can be in any ratio suitable for the formation of a co-complex. In certain embodiments, the enterostatin and naphthoic acid are in the ratio of 1:100, 1:10, 1:5, 1:4, 1:3, 1:2, 1:1.5, 1:1.25, 1:1.2, 1:1.1, 1.1:1, 1.2:1, 1.25:1, 1.5:1, 2:1, 3:1, 4:1, 5:1, 10:1 or 100:1 enterostatin:naphthoic acid. Ratios close to 1:1 are preferred.

Although the final solid forms of the invention can have reduced hygroscopicity, preparation of the solid forms themselves can be advantageous to reduce the amount of water in the final form. Accordingly, in some embodiments, the solid form is prepared under anhydrous conditions. In some embodiments, the host molecule, or the guest molecule, or both, is prepared under anhydrous conditions. However, the present invention is in no way limited by the method of preparation of the solid forms. Accordingly, the present invention also provides methods of preparing the solid forms without regard to hydrous or anhydrous conditions.

Once prepared, the enterostatin co-complexes can be stored under any conditions for the storage of a peptide complex known to those of skill in the art. Although the co-complexes can display advantageous hygroscopicity, in preferred embodiments the co-complexes are stored at low humidity conditions to maximize the stability of the co-complexes.

### 5.5 Methods of Treatment or Prevention

The enterostatin co-complexes of the invention can be used for the treatment or prevention of any disorder or condition amenable to treatment with enterostatin according to the judgment of those of skill in the art. The condition can be associated with normal or abnormal enterostatin function. For instance, in certain embodiments, an enterostatin co-complex of the invention can be administered to a subject that expresses or secretes a low amount of enterostatin to reduce or ameliorate any symptom of the low amount of enterostatin. Such methods of treatment are described in co-pending U.S. provisional application No. 60/750,208, filed Dec. 13, 2005, the contents of which are hereby incorporated by reference in their entirety.

In certain embodiments, the co-complexes of the invention can be used for the treatment or prevention of metabolic conditions or dyslipidemic conditions. Exemplary disorders or conditions related to enterostatin or F₁-ATPase activity include, but are not limited to, overweight, obesity, metabolic disorders, hypertension, lipid related disorders, and type II diabetes.

### 5.6 Compositions and Methods of Administration

The present invention provides co-complexes of enterostatin that are useful for the preparation of a pharmaceutical composition that can be used, for example, for the treatment of conditions associated with enterostatin function.

In a specific embodiment, a composition comprises one or more co-complexes of the invention, or a pharmaceutically acceptable salt, solvate, or hydrate thereof.

In a preferred embodiment, a composition of the invention is a pharmaceutical composition or a single unit dosage form. Pharmaceutical compositions and single unit dosage forms of the invention comprise a prophylactically or therapeutically effective amount of one or more prophylactic or therapeutic agents (e.g., a co-complex of the invention, or other prophylactic or therapeutic agent), and a typically one or more pharmaceutically acceptable carriers or excipients. In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical pharmaceutical compositions and dosage forms comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a patient and the specific active ingredients in the dosage form. The composition or single unit dosage form, if desired, can also contain minor amounts of wetting or emulsifying agents, or pH buffering agents.

Lactose-free compositions of the invention can comprise excipients that are well known in the art and are listed, for example, in the U.S. Pharmocopia (USP) SP (XXI)/NF (XVI). In general, lactose-free compositions comprise an active ingredient, a binder/filler, and a lubricant in pharmaceutically compatible and pharmaceutically acceptable amounts. Exemplary lactose-free dosage forms comprise an active ingredient, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

The pharmaceutical compositions and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a prophylactic or therapeutic agent preferably in purified form, together with a suitable amount of carrier so as to provide the form for proper administration to the patient. The formulation should suit the mode of administration. In a preferred embodiment, the pharmaceutical compositions or single unit dosage forms are sterile and in suitable form for administration to a subject, preferably an animal subject, more preferably a mammalian subject, and most preferably a human subject.

A pharmaceutical composition of the invention is formulated to be compatible with its intended route of administration. Examples of routes of administration include, but are not limited to, parenteral, e.g., intravenous, intradermal, subcutaneous, intramuscular, subcutaneous, oral, buccal, sublingual, inhalation, intranasal, transdermal, topical, transmucosal, intra-tumoral, intra-synovial and rectal administration. In a specific embodiment, the composition is formulated in accordance with routine procedures as a pharmaceutical composition adapted for intravenous, subcutaneous, intramuscular, oral, intranasal or topical administration to human beings. In an embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of inflammation or a related disorder may contain larger amounts of one or more of the active ingredients it comprises than a dosage form used in the chronic treatment of the same disease. Also, the therapeutically effective dosage form may vary among different types of cancer. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Typical dosage forms of the invention comprise a co-complex of the invention, or a pharmaceutically acceptable salt, solvate or hydrate thereof lie within the range of from about 0.1 mg to about 1000 mg per day, given as a single once-a-day dose in the morning but preferably as divided doses throughout the day taken with food. Particular dosage forms of the invention have about 0.1, 0.2, 0.3, 0.4, 0.5, 1.0, 2.0, 2.5, 5.0, 10.0, 15.0, 20.0, 25.0, 50.0, 100, 200, 250, 500 or 1000 mg of the active enterostatin.

5.6.1.1 Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

In preferred embodiments, the oral dosage forms are solid and prepared under anhydrous conditions with anhydrous ingredients, as described in detail in the sections above. However, the scope of the invention extends beyond anhydrous, solid oral dosage forms. As such, further forms are described herein.

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

5.6.1.2 Delayed Release Dosage Forms

Active ingredients such as the co-complexes of the invention can be administered by controlled release means or by delivery devices that are well known to those of ordinary skill in the art. Examples include, but are not limited to, those described in U.S. Pat. Nos.: 3,845,770; 3,916,899; 3,536,809; 3,598,123; and 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500 and 6,740,634, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

5.6.1.3 Transdermal, Topical & Mucosal Dosage Forms

Although solid, anhydrous oral dosage forms are preferred, the present invention also provides transdermal, topical, and mucosal dosage forms. Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

5.6.1.4 Dosage & Frequency of Administration

The amount of the co-complex or composition of the invention which will be effective in the prevention, treatment, management, or amelioration of a disorder or one or more symptoms thereof will vary with the nature and severity of the disease or condition, and the route by which the active ingredient is administered.. The frequency and dosage will also vary according to factors specific for each patient depending on the specific therapy (e.g., therapeutic or prophylactic agents) administered, the severity of the disorder, disease, or condition, the route of administration, as well as age, body, weight, response, and the past medical history of the patient. Effective doses may be extrapolated from dose-response curves derived from in vitro or animal model test systems.

Exemplary doses of a co-complex include milligram or microgram amounts of the active peptide per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). For co-complexes of the invention, the dosage administered to a patient is typically 0.01 mg/kg to 15 mg/kg of the patient's body weight, based on weight of the active peptide. Preferably, the dosage administered to a patient is between 0.01 mg/kg and 15 mg/kg, 0.01 mg/kg and 10 mg/kg, 0.01 mg/kg and 5 mg/kg, 0.01 and 4 mg/kg, 0.01 and 3 mg/kg, 0.01 mg/kg and 2 mg/kg, 0.01 mg/kg and 1 mg/kg, 0.02 mg/kg and 1 mg/kg, 0.10 mg/kg and 2.5 mg/kg, of the patient's body weight.

In general, the recommended daily dose range of a co-complex of the invention for the conditions described herein lie within the range of from about 0.01 mg to about 1000 mg of the active peptide per day, as a single dose or multiple doses per day. Specifically, a total daily dose range should be from about 1 mg to about 500 mg per day, more specifically, between about 10 mg and about 200 mg per day. In managing the patient, the therapy can be initiated at a lower dose, perhaps about 1 mg to about 25 mg, and increased if necessary up to about 200 mg to about 1000 mg per day as either a single dose or divided doses, depending on the patient's global response. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response. In certain embodiments, a co-complex or composition of the invention is administered in an amount of about 1 mg/day to about 500 mg/day of the active peptide. In some embodiments, it is administered in an amount of about 1 mg/day to about 400 mg/day of the active peptide. In some embodiments, it is administered in an amount of about 1 mg/day to about 300 mg/day of the active peptide. In some embodiments, it is administered in an amount of about 1 mg/day to about 200 mg/day of the active peptide. In some embodiments, it is administered in an amount of about 1 mg/day to about 100 mg/day of the active peptide.

A co-complex or composition of the invention can be administered as a single once-a-day dose or preferably as divided doses throughout a day. In some embodiments, the daily dose is administered twice daily in equally divided doses. In other embodiments, the daily dose is administered three times per day. In particular embodiments, the daily dose is administered three times per day in equally divided doses. In some embodiments, the daily dose is administered three times per day in three divided doses and each dose comprises the active peptide in an amount between about 1-100 mg, about 4-60 mg, about 4-40 mg, about 4-30 mg, about 4-25 mg, or about 4-20 mg. Preferably, the three divided doses of the co-complex are given around three meal times each day.

A co-complex or composition of the invention can be administered at various times. In some embodiments, it is administered to an enterostatin-deficient subject when the subject is fasted. In some embodiments, it is administered prior to a meal. In some embodiments, it is administered during a meal. In some embodiments, it is administered after a meal.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the co-complexes of the invention are also encompassed by the above described dosage amounts and dose frequency schedules. Further, when a patient is administered multiple dosages of a co-complex of the invention, not all of the dosages need be the same. For example, the dosage administered to the patient may be increased to improve the prophylactic or therapeutic effect of the co-complex or it may be decreased to reduce one or more side effects that a particular patient is experiencing.

In a specific embodiment, the dosage of the co-complex of the invention or a co-complex of the invention, based on weight of the active peptide, administered to prevent, treat, manage, or ameliorate a disorder, or one or more symptoms thereof in a patient is 0.01 mg/kg, 0.05 mg/kg, 0.10 mg/kg, 0.15 mg/kg, 0.20 mg/kg, 0.25 mg/kg, 0.5 mg/kg, 0.75 mg/kg, 1 mg/kg, 1.5 mg/kg, 2 mg/kg, 3 mg/kg, 4 mg/kg, 5 mg/kg, 10 mg/kg, or 15 mg/kg or more of a patient's body weight. In another embodiment, the dosage of the co-complex of the invention or a co-complex of the invention administered to prevent, treat, manage, or ameliorate a disorder, or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg mg, 0.1 mg to 5 mg, 0.1 to 2.5 mg, 0.25 mg to 20 mg, 0.25 to 15 mg, 0.25 to 12 mg, 10 mg, 0.25 to 8 mg, 0.25 mg to 7 mg, 0.25 mg to 5 mg, 0.5 mg to 2.5 mg, 1 mg to 1 mg to 15 mg, 1 mg to 12 mg, 1 mg to 10 mg, 1 mg to 8 mg, 1 mg to 7 mg, 1 mg to 5 mg, or 1 mg to 2.5 mg.

In certain embodiments, administration of the same co-complex of the invention may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In certain embodiments, the co-complex of the invention or a co-complex of the invention can be administered as a single, one time dose or chronically. By chronic it is meant that the co-complex of the invention or a co-complex of the invention is practiced more than once to a given individual. For example, chronic administration can be multiple doses of a pharmaceutical composition administered to a subject, on a daily basis, twice daily basis, or more or less frequently, as will be apparent to those of skill in the art. Chronic administration can continue for days, weeks, months or years if appropriate according to the judgment of the practitioner of skill.

In another embodiment, the co-complex of the invention or a co-complex of the invention is administered acutely. By acute it is meant that the co-complex of the invention or a co-complex of the invention is administered in a time period close to or contemporaneous with the onset of an event. For example, acute administration can be a single dose or multiple doses of a pharmaceutical composition administered around the onset of a meal. In some embodiments, the meal is a high calorie or high fat meal. Acute administration can also be a single dose or multiple doses of a pharmaceutical composition administered around the onset of a craving for food, specifically a craving for fatty food. A time period close to or contemporaneous with the onset of an event will vary according to the event but can be, for example, within about 30 minutes of a meal or a craving for food. In certain embodiments, acute administration is administration within about an hour of a meal or a craving for food. In certain embodiments, acute administration is administration within about 2 hours, about 6 hours, about 10 hours, about 12 hours, about 15 hours or about 24 hours after a meal or a craving for food.

In a specific embodiment, the invention provides a method of preventing, treating, managing, or ameliorating a disorder, or one or more symptoms thereof, said methods comprising administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more co-complexes of the invention once every 3 days, preferably, once every 4 days, once every 5 days, once every 6 days, once every 7 days, once every 8 days, once every 10 days, once every two weeks, once every three weeks, or once a month.

The following synthetic and biological examples are offered to illustrate this invention and are not to be construed in any way as limiting the scope of this invention.

### 6. EXAMPLES

Analytical techniques discussed below were conventional unless otherwise specified. In particular, differential scanning calorimetry and moisture balance experiments were carried out according to standard techniques. NMR was in D₂O.

### 6.1 Example 1

### Formation of Co-complexes

The instant example illustrates methods of producing co-complexes of the invention.

Enterostatin cocrystals were prepared by combining a cocrystal guest and an enterostatin salt in a 1:1 molar ratio in a solvent. The solvents were allowed to evaporate and the resulting solid co-complex was collected.

### 6.2 Example 2

### Enterostatin Acetate, 1-Hydroxy-2-naphthoic Acid

The instant example provides a co-complex comprising enterostatin acetate and 1-hydroxy-2-naphthoic acid.

The co-complex was prepared according to Example 1 in the solvent methanol. The salt was enterostatin acetate, and the guest was 1-hydroxy-2-naphthoic acid. The resulting solid was in the form of light brown flakes or broken glass.

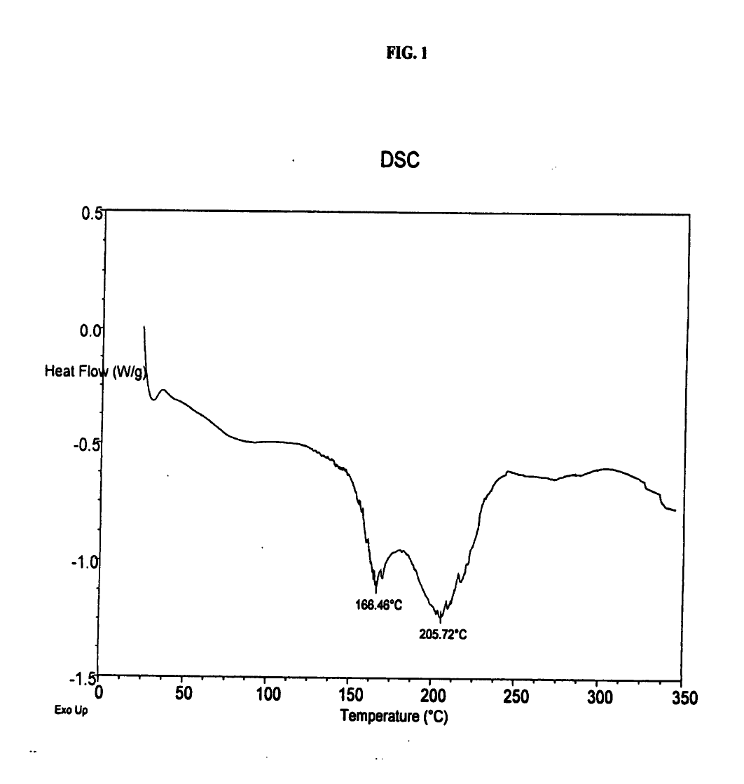

FIG. 1 provides differential scanning calorimetry (DSC) measurements of this enterostatin acetate, 1-hydroxy-2-naphthoic acid co-complex of the invention. The DSC curve has two broad endotherms at 167° C. and at 206° C.

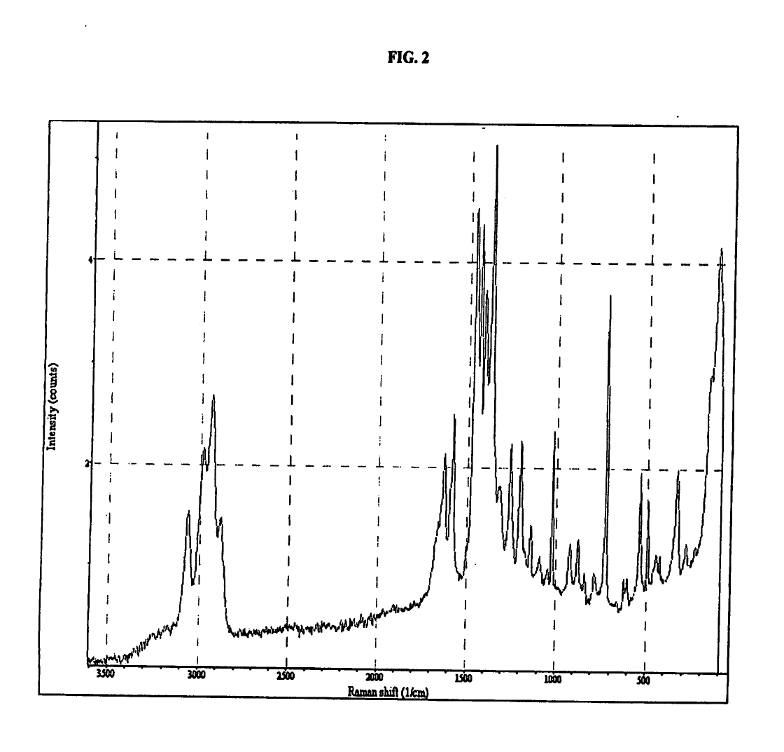

FIG. 2 provides a Raman spectrum of this enterostatin acetate, 1-hydroxy-2-naphthoic acid co-complex of the invention.

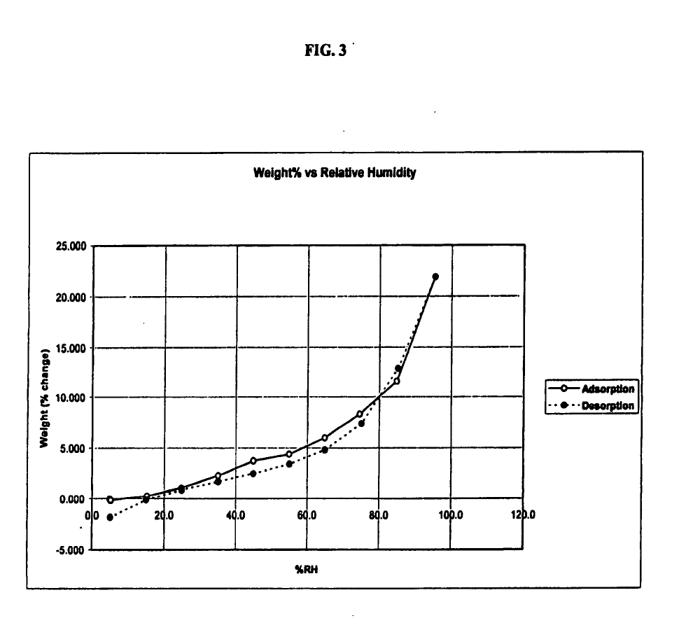

FIG. 3 provides moisture balance of this enterostatin acetate, 1-hydroxy-2-naphthoic acid co-complex of the invention. The material shows a 22% weight gain from 5 to 95% relative humidity and a 24% weight loss from 95 to 5% relative humidity.

NMR analysis of this enterostatin acetate, 1-hydroxy-2-naphthoic acid co-complex of the invention was consistent with a 1:1 complex.

A sample of this enterostatin acetate, 1-hydroxy-2-naphthoic acid co-complex of the invention was stressed at 58% relative humidity for 4 or 10 days. This enterostatin acetate, 1-hydroxy-2-naphthoic acid co-complex of the invention remained solid at 58% humidity for 4 or 10 days.

### 6.3 Example 3

### Enterostatin Chloride, 1-Hydroxy-2-naphthoic Acid

The instant example provides a co-complex comprising enterostatin chloride and 1-hydroxy-2-naphthoic acid.

The co-complex was prepared according to Example 1 in the solvent methanol. The salt was enterostatin chloride, and the guest was 1-hydroxy-2-naphthoic acid. The resulting solid was in the form of light brown fragments or broken glass.

NMR analysis of this enterostatin chloride, 1-hydroxy-2-naphthoic acid co-complex of the invention was consistent with less than a 1:1 complex.

A sample of this enterostatin chloride, 1-hydroxy-2-naphthoic acid co-complex of the invention was stressed at 58% relative humidity for 4 or 10 days. This enterostatin chloride, 1-hydroxy-2-naphthoic acid co-complex of the invention remained solid at 58% humidity for 4 or 10 days.

### 6.4 Example 4

### Enterostatin Acetate, Gentisic Acid

The instant example provides a co-complex comprising enterostatin acetate and gentisic acid.

The co-complex was prepared according to Example 1 in the solvent methanol. The salt was enterostatin acetate, and the guest was gentisic acid. The resulting solid was in the form of chunks or broken glass.

NMR analysis of this enterostatin acetate, gentisic acid co-complex of the invention was consistent with a 1:1 complex.

### 6.5 Example 5

### Enterostatin Chloride, Camphoric Acid

The instant example provides a co-complex comprising enterostatin chloride and camphoric acid.

The co-complex was prepared according to Example 1 in the solvent methanol. The salt was enterostatin chloride, and the guest was camphoric acid. The resulting solid was in the form of jagged flakes.

NMR analysis of this enterostatin chloride, camphoric acid co-complex of the invention was consistent with a 1:1 complex.

### 6.6 Example 6

### Enterostatin Sulfate, Piperazine

The instant example provides a co-complex comprising enterostatin sulfate and piperazine.

The co-complex was prepared according to Example 1 in the solvent hexafluoroispropanol. The salt was enterostatin sulfate, and the guest was piperazine. The resulting solid was in the form of white bits of solid material.

The co-complex was prepared a second time in the solvent hexafluoroispropanol with refrigeration and centrifugation. The resulting solid was a small amount of white solid.

NMR analysis of this enterostatin sulfate, piperazine co-complex of the invention was consistent with a greater than a 1:1 complex.

### 6.7 Example 7 1032

### Enterostatin Sulfate, Hippuric Acid

The instant example provides a co-complex comprising enterostatin sulfate and hippuric acid.

The co-complex was prepared according to Example 1 in the solvent hexafluoroispropanol. The salt was enterostatin sulfate, and the guest was hippuric acid. The resulting solid was in the form of solid fragments or broken glass.

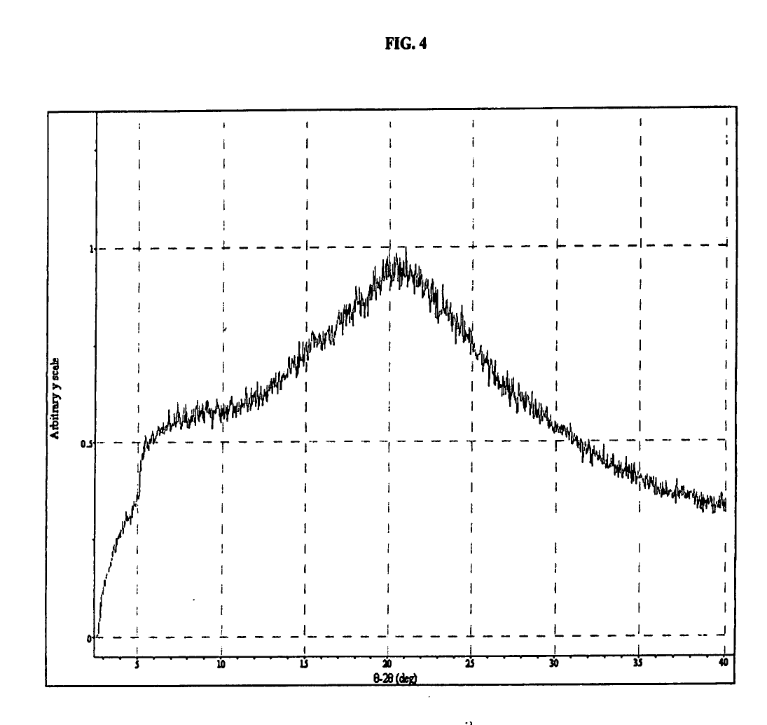

FIG. 4 provides an X-ray powder diffraction (XRPD) analysis of this enterostatin sulfate, hippuric acid co-complex of the invention. The XRPD peaks indicate a physical mixture of solid hippuric acid and solid enterostatin sulfate.

NMR analysis of this enterostatin sulfate, hippuric acid co-complex of the invention was consistent with a 1:1 complex.

### 6.8 Example 8

### Enterostatin Phosphate, Piperazine

The instant example provides a co-complex comprising enterostatin phosphate and piperazine.

The co-complex was prepared according to Example 1 in the solvent hexafluoroispropanol. The salt was enterostatin phosphate, and the guest was piperazine. The resulting solid was in the form of white solid fragments.

NMR analysis of this enterostatin phosphate, piperazine co-complex of the invention was consistent with greater than a 1:1 complex.

### 6.9 Example 9

### Enterostatin Phosphate, Adipic Acid

The instant example provides a co-complex comprising enterostatin phosphate and adipic acid.

The co-complex was prepared according to Example 1 in the solvent hexafluoroispropanol. The salt was enterostatin phosphate, and the guest was adipic acid. The resulting solid was in the form of solid fragments.

NMR analysis of this enterostatin phosphate, adipic acid co-complex of the invention was consistent with greater than a 1:1 complex.

### 6.10 Example 10

### Humidity Stress Experiments

A sample of each co-complex of the above examples was stressed at 58% relative humidity for either 4 or 10 days. Both the enterostatin acetate and HCl with 1-OH-2-napthoic acid as the guest (Examples 2 and 3, respectively) remained solid after 4 or 10 days. The co-complex comprising enterostatin sulfate and piperazine as the guest (Example 6) was a white film after 4 days. The other co-complexes resulted in either tacky or deliquesced material.

The cocrystal guests piperazine and 1-OH-2-napthoic acid were stressed at 58% relative humidity. Piperazine resulted in an oil puddle after 5 or 14 days. 1-OH-2-napthoic remained a solid after 14 days.

| Co-Complex or Control | Conditions | Observations |
|---|---|---|
| Example 2 | 58% RH, 4 days | Bits, broken glass |
| Example 3 | 58% RH, 4 days | Irregulars, chunks |
| Example 4 | 58% RH, 10 days | Deliquesced |
| Example 5 | 58% RH, 10 days | Deliquesced |
| Example 6 | 58% RH, 4 days | Transparent white film |
| Example 7 | 58% RH, 10 days | Tacky white goo |
| Example 8 | 58% RH, 4 days | Rounded thick tacky solid |
| Example 9 | 58% RH, 10 days | Tacky white goo |
| Piperazine | 58% RH, 14 days | Oil puddle |
| | 58% RH, 5 days | Oil puddle |
| 1-OH-2-napthoic acid | 58% RH, 14 days | Light brown chunks |

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A non-hygroscopic, stable solid form of enterostatin.

2. The solid form of claim 1 further comprising a guest molecule, or a salt or solvate thereof.

3. The solid form of claim 1 further wherein said enterostatin is in the form of a salt or solvate.

4. The solid form of claim 1 wherein said enterostatin is stable at about 4° C. for at least two years.

5. The solid form of claim 1 wherein said enterostatin is stable at about ambient temperature for at least two years.

6. The solid form of claim 1 having a water content of between about 0 weight percent and about 20 weight percent.

7. The solid form of claim 1 having a water content of between about 0 weight percent and about 15 weight percent.

8. The solid form of claim 1 having a water content of between about 0.5 weight percent and about 6 weight percent.

9. The solid form of claim 1 wherein said enterostatin is a peptide having an amino acid selected from the group consisting of APGPR (SEQ ID NO: 1), VPDPR (SEQ ID NO:2) and VPGPR (SEQ ID NO:3).

10. The solid form of claim 1 wherein said enterostatin is a peptide having amino acid sequence APGPR (SEQ ID NO:1).

11. The solid form of claim 1 wherein said enterostatin is a peptide having amino acid sequence VPDPR (SEQ ID NO:2).

12. The solid form of claim 1 wherein said enterostatin is a peptide having amino acid sequence VPGPR (SEQ ID NO:3).

13. The solid form of claim 1 wherein said guest molecule is selected from the group consisting of adipic acid, piperazine, hippuric acid, piperazine, camphoric acid, gentisic acid, naphthoic acid.

14. The solid form of claim 1 wherein said guest molecule is adipic acid.

15. The solid form of claim 1 wherein said guest molecule is piperazine.

16. The solid form of claim 1 wherein said guest molecule is hippuric acid.

17. The solid form of claim 1 wherein said guest molecule is piperazine.

18. The solid form of claim 1 wherein said guest molecule is camphoric acid.

19. The solid form of claim 1 wherein said guest molecule is gentisic acid.

20. The solid form of claim 1 wherein said guest molecule is naphthoic acid.

21. The solid form of claim 1 wherein said guest molecule is 1-hydroxy-2-naphthoic acid.

22. The solid form of claim 1 comprising a solvate of the enterostatin.

23. The solid form of claim 1 comprising a hydrate of the enterostatin.

24. The solid form of claim 1 comprising an enterostatin salt.

25. The solid form of claim 24 wherein said enterostatin salt is selected from the group consisting of enterostatin chloride, enterostatin acetate, enterostatin sulfate and enterostatin phosphate.

26. The solid form of claim 24 wherein said enterostatin salt is enterostatin chloride.

27. The solid form of claim 24 wherein said enterostatin salt is enterostatin acetate.

28. The solid form of claim 24 wherein said enterostatin salt is enterostatin sulfate.

29. The solid form of claim 24 wherein said enterostatin salt is enterostatin phosphate.

30. The solid form of claim 24 comprising a solvate of the enterostatin salt.

31. The solid form of claim 24 comprising a hydrate of the enterostatin salt.

32. The solid form of claim 1 that adsorbs less than 30% water, by weight, from 5 to 95% relative humidity.

33. The solid form of claim 1 that desorbs less than 30% water, by weight, from 95 to 5% relative humidity.

34. The solid form of claim 1 that adsorbs less than 30% water, by weight, from 5 to 95% relative humidity and that desorbs less than 30% water, by weight, from 95 to 5% relative humidity.

35. A non-hygroscopic, stable solid form of enterostatin comprising an enterostatin salt and a guest molecule, or a salt or solvate thereof.

36. The solid form of claim 35 wherein said enterostatin is a peptide having an amino acid selected from the group consisting of APGPR (SEQ ID NO:1), VPDPR (SEQ ID NO:2) and VPGPR (SEQ ID NO:3).

37. The solid form of claim 35 wherein said guest molecule is selected from the group consisting of adipic acid, piperazine, hippuric acid, piperazine, camphoric acid, gentisic acid, naphthoic acid.

38. The solid form of claim 35 wherein said guest molecule is naphthoic acid.

39. The solid form of claim 35 wherein said guest molecule is 1-hydroxy-2-naphthoic acid.

40. The solid form of claim 35 wherein said enterostatin salt is selected from the group consisting of enterostatin chloride, enterostatin acetate, enterostatin sulfate and enterostatin phosphate.

41. The solid form of claim 35 comprising a solvate of the enterostatin salt.

42. The solid form of claim 35 comprising a hydrate of the enterostatin salt.

43. A crystalline, non-hygroscopic form of enterostatin.

44. The crystalline, non-hygroscopic form of enterostatin of claim 43 further comprising a guest molecule, or a salt or solvate thereof.

45. The crystalline, non-hygroscopic form of enterostatin of claim 43 wherein said enterostatin is in the form of a salt or solvate.

46. The crystalline, non-hygroscopic form of enterostatin of claim 43 wherein said enterostatin is a peptide having an amino acid selected from the group consisting of APGPR (SEQ ID NO:1), VPDPR (SEQ ID NO:2) and VPGPR (SEQ ID NO:3).

47. The crystalline, non-hygroscopic form of enterostatin of claim 43 wherein said guest molecule is selected from the group consisting of adipic acid, piperazine, hippuric acid, piperazine, camphoric acid, gentisic acid, naphthoic acid.

48. The crystalline, non-hygroscopic form of enterostatin of claim 43 comprising a solvate of the enterostatin.

49. The crystalline, non-hygroscopic form of enterostatin of claim 43 comprising an enterostatin salt.

50. The crystalline, non-hygroscopic form of enterostatin of claim 49 wherein said enterostatin salt is selected from the group consisting of enterostatin chloride, enterostatin acetate, enterostatin sulfate and enterostatin phosphate.

51. The crystalline, non-hygroscopic form of enterostatin of claim 49 comprising a solvate of the enterostatin salt.

52. An enterostatin co-complex comprising enterostatin and naphthoic acid, or one or more salts thereof.

53. The enterostatin co-complex of claim 52 that comprises an enterostatin with an amino acid sequence selected from the group consisting of APGPR (SEQ ID NO:1), VPDPR (SEQ ID NO:2) and VPGPR (SEQ ID NO:3).

54. The enterostatin co-complex of claim 52 that comprises an acetate or a chloride salt of enterostatin.

55. The enterostatin co-complex of claim 52 wherein said naphthoic acid is hydroxy-naphthoic acid.

56. The enterostatin co-complex of claim 52 wherein said naphthoic acid is according to formula (I): 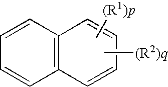
wherein each p and q is independently an integer from 1 to 3;
each R¹ is independently hydroxy or hydrogen; and
each R² is independently carboxyl;
wherein each R¹ or R2 can be on either ring of formula (I).

57. The enterostatin co-complex of claim 56 wherein p is 1 and q is 1.

58. The enterostatin co-complex of claim 56 wherein said naphthoic acid is 1-hydroxy-2-naphthoic acid.

59. The enterostatin co-complex of claim 56 that comprises enterostatin acetate, having the sequence APGPR (SEQ ID NO:1), and 1-hydroxy-2-naphthoic acid.

60. The enterostatin co-complex of claim 56 that comprises enterostatin chloride, having the sequence APGPR (SEQ ID NO:1), and 1-hydroxy-2-naphthoic acid.

61. A pharmaceutical composition comprising an enterostatin form according to claim 1, 35, 43, 52, 59 or 60, and a pharmaceutically acceptable carrier, excipient or diluent.

62. A method of treating or preventing a condition related to enterostatin deficiency, comprising the step of administering to a subject in need thereof an effective amount of a pharmaceutical composition according to claim 61.

63. The method of claim 62 wherein said condition is selected from the group consisting of overweight, obesity, hypertension, dyslipidemia, type 2 diabetes, coronary heart disease, stroke, gallbladder disease, osteoarthritis, sleep apnea and respiratory problems and cancer.

64. The method of claim 63 wherein said condition is obesity.

65. A method of suppressing appetite for fat in a subject in need thereof, comprising the step of administering to the subject an effective amount of a pharmaceutical composition according to claim 61.


SEQUENCE LISTING (3 sequences)

SEQ ID NO: 1 - protein, 5 aa, Homo sapiens
APGPR

SEQ ID NO: 2 - protein, 5 aa, Rat
VPDPR

SEQ ID NO: 3 - protein, 5 aa, Rat
VPGPR